(19) United States
(12) Patent Application Publication
(10) Pub. No.: US 2026/0308101 A1
(43) Pub. Date: Oct. 8, 2026

(54) METHOD FOR PURIFYING LIGHT ACRYLATES

(71) Applicant: Arkema France, Puteaux (FR)

(72) Inventors: Kevin COULET, Saint Avold Cedex (FR); Serge TRETJAK, Saint Avold Cedex (FR)

(21) Appl. No.: 19/481,904

(22) PCT Filed: May 15, 2024
(86) PCT No.: PCT/FR2024/050619

§ 371 Date: Nov. 6, 2025

(30) Foreign Application Priority Data
May 22, 2023 (FR) FR23.05029

(51) Int. Cl. C07C 67/54

(52) U.S. Cl. CPC C07C 67/54

(57) ABSTRACT

The present invention relates to the production of light (meth)acrylic esters, such as methyl or ethyl (meth)acrylate, by direct esterification of (meth)acrylic acid by the corresponding light alcohol. A subject matter of the invention is more particularly a process for the recovery/purification of C₁-C₂ alkyl acrylate comprising steps of evaporation and of partial condensation making it possible to remove a fraction rich in alkyl alkoxypropionate byproduct, which cannot be upgraded by a cracking process and which is very difficult to remove whether this is when it is pure, because it is close in boiling point to that of acrylic acid, or when it is in the form of an azeotrope with water, because it is close in boiling point to that of the acrylic ester.

### TECHNICAL FIELD

The present invention relates to the production of light (meth)acrylic esters, such as methyl or ethyl (meth)acrylate, by direct esterification of (meth)acrylic acid by the corresponding light alcohol.

A subject matter of the invention is more particularly a process for the recovery/purification of C₁-C₂ alkyl acrylate comprising steps of evaporation and of partial condensation making it possible to remove a fraction rich in alkyl alkoxypropionate byproduct, which cannot be upgraded by a cracking process and which is very difficult to remove whether this is when it is pure, because it is close in boiling point to that of acrylic acid, or when it is in the form of an azeotrope with water, because it is close in boiling point to that of the acrylic ester.

### PRIOR ART AND TECHNICAL PROBLEM

It is known to produce (meth)acrylic esters by carrying out an esterification reaction between an alcohol and a (meth)acrylic acid. This reaction is an equilibrated catalyzed reaction with generation of water. It is furthermore accompanied by side reactions which produce impurities.

It is necessary to remove the water produced in order to displace the equilibrium, to remove the impurities, as well as to recycle the unreacted reactants.

To this end, a set of distillations and/or of extractions, separations by settling, is generally carried out, which is both relatively complex to implement, in particular as a result of the presence of azeotropic mixtures, and costly in terms of energy.

The problems which arise during the manufacture of light (meth)acrylic esters, in particular C₁-C₂ alkyl acrylic esters, will now be set out, for convenience, on the basis of the example of methyl acrylate obtained by esterification of acrylic acid by methanol. However, the problems and the solution proposed by the invention can be applied to the use of ethanol in the esterification reaction.

By way of side reactions during the manufacture of methyl acrylate, unreacted acrylic acid can form oligomers, such as 3-acryloyloxypropionic acid (n=2) or 3-acryloyloxy-3-propoxypropionic acid (n=3), which are heavy byproducts with a higher boiling point than that of acrylic acid.

As another side reaction, a Michael addition can produce Michael adducts, in particular methyl methoxypropionate formed between methyl acrylate and methanol.

Methyl methoxypropionate (MMP) is a “heavy” byproduct which is formed in a significant amount in the process as the esterification reaction progresses, at the same time as the oligomers of acrylic acid. Its boiling point is close to that of acrylic acid (144° C., atmospheric P) but it can also form an azeotrope with water (97.3° C., atmospheric P) during the azeotropic distillation.

MMP is the lightest of the heavy byproducts compared to the acrylic acid oligomers; it can interfere in the final purification of the methyl acrylate (80° C., atmospheric P) and can detrimentally affect the quality of the finished product.

In order to limit the formation of MMP, it has been proposed in the document U.S. Pat. No. 6,025,520 to carry out the esterification reaction under reduced pressure with an excess of acid. These conditions make it possible to improve the yield and the selectivity of the esterification reaction and to significantly reduce the formation of heavy byproducts, such as MMP, problematic for the line for the purification of the desired ester.

The process described in the document WO 2015/063388 proposes to significantly reduce the formation of alkyl alkoxypropionate during the synthesis of methyl (meth)acrylate by carrying out the esterification reaction in a fixed bed reactor at atmospheric pressure and under conditions where the acid is in excess with respect to the alcohol and where the hourly space velocities are high.

In the document FR 3 083 233, the applicant company showed that it is possible to remove the MMP by sidestream drawoff during the azeotropic distillation of the reaction mixture carried out in a single distillation column provided with a sidestream drawoff. However, this solution remains problematic to implement because it requires very stable operating conditions, both in the esterification reaction and in the azeotropic distillation column.

There still exists a need to remove the methyl methoxypropionate, which is detrimental to the material balance of the process (loss of starting materials during the purges) and to the purification line (complexity to achieve a high purity).

One of the objectives of the present invention is thus to provide an improved process for the recovery/purification of methyl acrylate, and more generally of methyl or ethyl acrylate, making possible efficient removal of methyl methoxypropionate, by continuously carrying out an evaporation of the bottom product from the column for the purification of methyl acrylate and at least two successive condensations of the gas phase. A methyl acrylate stream, a methyl methoxypropionate stream and an inhibitor-rich evaporator bottom stream are thus obtained.

### SUMMARY OF THE INVENTION

A subject matter of the present invention is a process for the recovery/purification of C₁-C₂ alkyl acrylate from a reaction mixture resulting from the esterification of acrylic acid with an alcohol chosen from methanol and ethanol carried out in the presence of polymerization inhibitor(s), said process comprising at least the following steps:
the azeotropic distillation of said reaction mixture using a first distillation column, resulting in the separation:
   at the top, of a stream comprising the alkyl acrylate, the unreacted alcohol and water;
   at the bottom, of a fraction comprising the unreacted acrylic acid and heavy products,
the treatment of the top stream from the first distillation column by liquid/liquid extraction, generating an aqueous phase containing essentially alcohol and an organic phase rich in alkyl acrylate;
the treatment by distillation of said aqueous phase in a second distillation column, in order to separate:
   at the top, alcohol, which will be recycled in the esterification reaction, and
   at the bottom, an alcohol-depleted stream;
the treatment of the bottom stream from the first distillation column using a third distillation column and/or an evaporator, which separates:
   at the top, a stream comprising the residual acrylic acid and lighter products, and
   at the bottom, a stream consisting of heavy products;
the treatment of said organic phase rich in alkyl acrylate in a fourth distillation column, referred to as purification column, in order to separate:
   at the top, the purified alkyl acrylate and,
   at the bottom, the heavy products and the polymerization inhibitors,
the continuous treatment of the bottom stream from the purification column, by evaporation, and at least two successive condensations of the gas phase resulting from the evaporation, making it possible to obtain:
   an alkyl acrylate stream to be recycled to the purification column;
   an alkyl alkoxypropionate stream, and
   a residue containing the polymerization inhibitors.

This process makes it possible to minimize the alkyl acrylate losses and to recycle the polymerization inhibitors contained at different points in the process while removing a concentrated fraction rich in alkyl alkoxypropionate byproduct.

According to various embodiments, said process comprises the following features, if appropriate combined. The contents indicated are expressed by weight, unless otherwise indicated. In the ranges of values indicated, the limits are included.

The term azeotropic distillation is understood to mean the separation of an azeotrope (or azeotropic mixture) consisting of a ternary alkyl acrylate/alcohol/water mixture.

According to one embodiment, the alcohol is methanol, the alkyl acrylate is methyl acrylate, and the alkyl alkoxypropionate is methyl methoxypropionate (MMP).

According to one embodiment, the alcohol is ethanol, the alkyl acrylate is ethyl acrylate, and the alkyl alkoxypropionate is ethyl ethoxypropionate (EEP).

According to one embodiment, the azeotropic distillation column can be provided with a sidestream drawoff.

According to one embodiment, the azeotropic distillation column has no sidestream drawoff.

According to one embodiment, the azeotropic distillation is carried out by two distillation columns in series.

According to one embodiment, the final purification, in order to obtain commercial grade alkyl acrylate (purity of greater than 99.5% by weight), is carried out by employing at least one purification column, such as a tailing column and a topping column.

According to one embodiment, said at least one purification column is a dividing wall column (DWC).

According to one embodiment, said at least one purification column is a column having sidestream drawoff.

According to one embodiment, the evaporation step is carried out by means of a thin film evaporator.

According to one embodiment, the treatment of the bottom stream from the first distillation column is carried out by means of a thin film evaporator.

According to one embodiment, when the alkyl acrylate is methyl acrylate, methyl acrylate having a purity of greater than 99.5% by weight is obtained in a distillation column referred to as tailing column or a dividing wall column operating in a pressure range from 10 000 Pa to 50 000 Pa and a temperature range extending from 80° C. to 110° C.

According to one embodiment, the process according to the invention comprises the following steps:
a) azeotropic distillation of the reaction mixture using a first distillation column making it possible to separate, at the top, an azeotropic mixture comprising the alkyl acrylate, the unreacted alcohol and water and, at the bottom, a fraction comprising the unreacted acrylic acid and heavy byproducts,
b) separation of the bottom stream from the first distillation column into a stream comprising essentially the unreacted acrylic acid, this screen being, in all or part, recycled in the esterification reactor,
c) liquid/liquid extraction of the top stream from the first distillation column by an aqueous stream, making it possible to separate an organic phase, comprising essentially the alkyl acrylate, and an aqueous phase,
d) purification of said organic phase by means of a separation assembly comprising at least one column for the purification of the alkyl acrylate, such as a tailing column and a topping column, a column having sidestream drawoff or a dividing wall column (DWC), making it possible to recover the purified alkyl acrylate at the top and, at the bottom, a stream containing the heavy byproducts, such as alkyl alkoxypropionate,
e) treatment of the bottom stream from this purification column on a thin film evaporator in order to recycle the alkyl acrylate to the purification column, to remove a final residue comprising the polymerization inhibitors and to send a stream highly concentrated in alkyl alkoxypropionate to the purge for incineration.

According to one embodiment, when the alkyl acrylate is methyl acrylate, the bottom stream from the purification column making it possible to obtain methyl acrylate is subjected to an evaporation and to successive condensations of the gas phase from the evaporator. This evaporation operation generates three streams:
a methyl acrylate stream to be recycled to the purification column;
an MMP stream, and
a residue stream containing the polymerization inhibitors which can advantageously be recycled in the process.

Pressure of the evaporator and reaction temperature are linked together so that the methyl acrylate and the MMP are removed by evaporation.

According to one embodiment, the evaporation is carried out in a temperature range extending from 80° C. to 110° C. and more especially between 90° C. and 100° C.

According to one embodiment, the maintained pressure of the evaporator is of between 10 000 Pa and 80 000 Pa.

According to one embodiment, the top stream from this evaporator is cooled in two successive steps:
partial condensation in a temperature range extending from 70° C. to 110° C., preferably between 80° C. and 90° C., at the same pressure as that of the evaporator, in order to obtain, on the one hand, a liquid stream of Michael adducts to be purged and, on the other hand, a vapor stream;
condensation of this vapor stream in a temperature range extending from 20° C. to 50° C., more especially from 30° C. to 40° C., before this liquid stream is taken up by a pump and fed to the purification column.

The composition by weight of the product for feeding the evaporator at the bottom of the purification column in the case of the manufacture of methyl acrylate in the presence of hydroquinone as polymerization inhibitor is as follows:
methyl acrylate (60-100%),
methyl methoxypropionate (MMP) 5-40%,
hydroquinone: 0.1 to 2%.

According to one embodiment, the fraction rich in alkyl alkoxypropionate contains alkyl alkoxypropionate at a content by weight which can range from approximately 20% to approximately 80%.

According to one embodiment, the reaction mixture results from the esterification of acrylic acid with a stoichiometric excess of alcohol.

According to one embodiment, the reaction mixture results from the esterification of acrylic acid with an alcohol under conditions of stoichiometric excess of acid.

According to one embodiment, the azeotropic distillation is carried out under a slight vacuum under a pressure which can range from 200 to 600 mmHg.

According to one embodiment, the temperature at the bottom of the azeotropic distillation column is less than 110° C., preferably less than 100° C.

The reaction is carried out in the presence of one or more polymerization inhibitors which are introduced into the reactor in a proportion of 500 to 5000 ppm, with respect to the crude reaction mixture. Mention may be made, as polymerization inhibitors which can be used, for example, of phenothiazine, hydroquinone, hydroquinone monomethyl ether, di(tert-butyl)-para-cresol (BHT), para-phenylenediamine, TEMPO (2,2,6,6-tetramethyl-1-piperidinyloxy), di(tert-butyl)catechol or TEMPO derivatives, such as HO-TEMPO, alone or their mixtures in all proportions. A supplementary addition of polymerization inhibitor is generally carried out at the subsequent purification treatment.

The effectiveness of the inhibitors is also related to the injection of air or of depleted air into the various columns employed.

According to one embodiment, the separation assembly comprises at least one column for the purification of the alkyl acrylate, such as a tailing column and a topping column.

According to one embodiment, the separation assembly comprises at least one column for the purification of the alkyl acrylate, such as a dividing wall column (DWC).

According to one embodiment, the separation assembly comprises at least one column for the purification of the alkyl acrylate, such as a column having sidestream drawoff.

According to one embodiment, the purification step d) is carried out by distillation, using a purification column comprising at least one distillation column, preferably two distillation columns in series.

The invention makes it possible to limit the losses of economically upgradable materials, such as acrylic acid, the alcohol or the alkyl acrylate, in a process for the production of light alkyl acrylate by direct esterification.

According to one embodiment, the final purification making it possible to obtain methyl acrylate can be carried out using a tailing column and a topping column.

The invention makes it possible to efficiently remove the alkyl alkoxypropionate and to reduce the losses of economically upgradable products generated by the purges imposed by the accumulation of alkyl alkoxypropionate in the purification line and to recycle the polymerization inhibitors advantageously in the process.

Thus, the invention provides a simplified process for the production of high purity methyl or ethyl acrylate, and optimizes the material balance of the process.

### BRIEF DESCRIPTION OF THE FIGURES

FIG. 1 diagrammatically represents a plant for producing methyl acrylate by employing the recovery/purification process according to the invention.

### DETAILED ACCOUNT OF THE INVENTION

The invention is now described in more detail and in a nonlimiting way in the description which follows. For simplicity, the description is based on the example of methyl acrylate obtained by esterification of acrylic acid by methanol. The solution proposed by the invention applies in the same way to the employment of ethanol in the esterification reaction, and also to other configurations of the purification line (process with an azeotropic distillation equipped with sidestream drawoff, dividing wall column for the final purification).

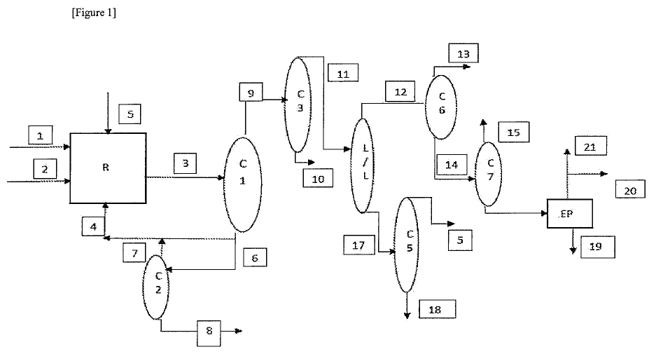

A plant for the production of methyl acrylate is represented in FIG. 1.

The reaction section comprises an esterification reactor R. The reactor R is fed by an acrylic acid feed pipe 1 and a methanol feed pipe 2. The reactor preferably contains a heterogeneous catalyst of acidic cation exchange resin type. In the case of homogeneous catalysis, the reactor is additionally fed by a catalyst feed pipe (not represented). The esterification reaction can be carried out in excess of methanol or in excess of acrylic acid.

At the outlet of the reactor R, the reaction mixture 3 is sent to an azeotropic distillation unit represented in FIG. 1 by two distillation columns in series C1 and C3, the first column C1 being connected to the second column C3 by the gas phase 9, and the second column C3 separating, at the bottom, a stream 10 of water consisting of a part of the water formed by the esterification reaction and of water injected in the form of steam into the column C3.

According to one embodiment, the azeotropic distillation unit comprises a single distillation column C1 operating under vacuum under the same conditions.

According to one embodiment, the azeotropic distillation unit comprises a distillation column equipped with a sidestream drawoff.

The distillation column C1 separates, at the bottom, a stream 6 comprising essentially unreacted acrylic acid, traces of light products (boiling point lower than that of acrylic acid), and heavy products having a higher boiling point than acrylic acid (oligomers of acrylic acid and Michael adducts).

The stream 6 is sent to a distillation column C2 which separates a stream 7, comprising the residual acrylic acid and the lighter products, and a stream 8 consisting essentially of the heavy products. The stream 7 is advantageously recycled in the reactor R.

The top stream 11 from the azeotropic distillation unit is sent to a liquid/liquid extraction section L/L (settling tank or contactor) which generates, on the one hand, an aqueous phase 17 containing essentially methanol and, on the other hand, an organic phase 12.

In the plant described in FIG. 1, the aqueous phase 17 is subjected to a distillation on a distillation column C5 in order to separate the methanol, which is recycled to the reactor (stream 5), and an aqueous stream 18 depleted in methanol which can be partially recycled in the liquid/liquid extraction phase.

The organic phase 12 can be subjected to one or more additional steps of distillation in series. For example, a topping column C6 extracts the residual light compounds 13 at the top and a tailing column C7 which separates, at the bottom, a stream 16 comprising essentially the residual heavy compounds with methyl acrylate and a purified stream 15 of methyl acrylate at the top.

According to one embodiment, the columns C6 and C7 can be replaced by a dividing wall column (DWC).

The stream concentrated in heavy compounds 16 is treated on a thin film evaporator. The bottom product 19 from the evaporator, which is rich in polymerization inhibitors, can be mixed and recycled with the stream 4. The gas phase resulting from this evaporator EP is partially condensed in order to obtain a first liquid stream 20 rich in Michael adducts and, after a second condensation, a stream 21 of methyl acrylate which can advantageously be recycled in the feed stream 14 of C7.

With reference to FIG. 1, the column C7 has an equivalent of 10 to 30 theoretical plates, preferably 10 to 15 theoretical stages, and operates in a pressure range from 10 000 Pa to 50 000 Pa and a temperature range extending from 80° C. to 110° C. at the column bottom. The internals used for the column can be valve trays or perforated trays having a weir, crossflow trays, such as dual flow trays, ripple trays or Shell Turbogrid trays, or stacked packing. The bottom stream is concentrated on a thin film evaporator EP, very well suited to viscous fouling products and to contaminated liquids. The evaporation is carried out in a temperature range extending from 90° C. to 110° C. and more especially between 90° C. and 100° C., in a pressure range of between 10 000 Pa and 80 000 Pa.

When the purification system employed comprises a dividing wall column according to WO2017/125637, the thin film evaporator will treat the same stream 16 at the bottom of the DWC column, generating a stream of polymerization inhibitors at the bottom, to be recycled at the inlet of the reactor R, and two liquid condensation products: MMP, which is purged and removed, and methyl acrylate, which is recycled in the feeding of the dividing wall column.

The methyl acrylate recovery/purification process according to the invention applies to a reaction mixture 3 resulting from the esterification of acrylic acid 1 with methanol 2 in the reactor R.

The following examples illustrate the present invention and do not have the aim of limiting the scope of the invention as defined by the appended claims.

### EXPERIMENTAL PART

In the examples, the percentages are indicated by weight, unless otherwise indicated, and the following abbreviations were used:
MA: methyl acrylate
MMP: methyl methoxypropionate
HQ: polymerization inhibitor
AA: acrylic acid
MeOH: methanol

### Example 1 According to the Invention

Simulations using a thermodynamic model were carried out for a reaction mixture obtained by reaction of methanol with AA in excess, on an acidic cationic resin.

The inlet flow rate of the reaction mixture in the purification line is adjusted in order to ensure a production of methyl acrylate MA of approximately 160 t/d.

The flow rate of methyl methoxypropionate MMP to be purged is of the order of 65 kg/h, corresponding to the amount of MMP formed in the reaction at the rate of 160 t/d.

Two configurations were compared; they differ in the implementation of the treatment of the column C7 bottom product:
a direct purge of the column C7 bottom product, making it possible to remove the MMP and to prevent it from accumulating, and
a treatment of the bottom product prior to the purging of the MMP, making it possible to limit the loss of MA and of polymerization inhibitor (HQ) according to the invention, employing a thin film evaporator and a partial condensation of the vapor.

A comparison is given in table 1 below of the distribution of the different flow rates for the purging of MMP, expressed in kg/h, in the two configurations: according to the prior art (Reference) and according to the invention.

TABLE 1

| | Reference | Invention |
|---|---|---|
| MMP (%) | 17.5 | 37.4 |
| MA (%) | 79.1 | 59.4 |
| Polymerization inhibitor (%) | 0.9 | 0.03 |

The material balance of the MMP purge point in each of the two processes, respectively the stream 16 at the bottom of the column C7 in the reference process and the bottom stream 20 from the first condenser of the EP section in the process according to the invention, is shown in table 2.

TABLE 2

| | Reference Purge | Invention Purge |
|---|---|---|
| Overall flow rate (kg/h) | 250 | 117 |
| MMP (kg/h) | 43.8 | 43.6 |
| MA (kg/h) | 198 | 69 |
| HQ (kg/h) | 2.1 | 0 |

In the reference process, the simulation indicates significant losses of MA, which is concentrated at 79% in the bottom of the tailing column C7. On the contrary, the process according to the invention makes it possible to limit the content of upgradable product in the purge.

### Comparative Example 2: Process for the Removal of MMP in the Sidestream Drawoff According to FR 3 083 233

The pilot plant used employs a distillation column filled with structured packing elements equivalent to 25 theoretical stages.

It is fed in the lower part with 10 kg/h of synthetic mixture (AA, MeOH, water, MMP). Reflux is provided by an external flow rate of 5.7 kg/h of MA and the sidestream drawoff is carried out (30 g/h) in the upper part. Polymerization inhibitors are injected at the top into the reflux and into the condenser and also into the feed.

For this study and in order to show the sensitivity of the performance qualities of this process to the operating conditions, the bottom drawoff flow rate was modified, while the flow rates and compositions of the feed and of the reflux which are external were kept constant.

The results have been combined in table 3.

TABLE 3

Bottom flow rate (kg/h)

| | 5 | 5.1 | 5.2 | 5.3 |
|---|---|---|---|---|
| % MMP SD | 38 | 35 | 30 | 20 |

A fluctuation of less than a few percent results in MMP purge performance qualities and makes this process problematic to operate. This sensitivity to the operating conditions of the sidestream drawoff does not occur in the case of the process according to the invention, since the composition and the flow rate of the stream 16 remain constant, which ensures a continuous performance quality of the evaporator EP.

1. A process for the recovery and/or purification of C1-C2 alkyl acrylate from a reaction mixture resulting from esterification of acrylic acid with an alcohol chosen from methanol and ethanol carried out in the presence of polymerization inhibitor(s), said process comprising the following steps:
azeotropic distillation of said reaction mixture in a first distillation column, resulting in the separation:
at the top of the first distillation column, of a top stream comprising the C1-C2 alkyl acrylate, unreacted alcohol and water;
at the bottom of the first distillation column, of a bottom fraction comprising unreacted acrylic acid and heavy products,
treatment of the top stream from the first distillation column by liquid/liquid extraction, generating an aqueous phase comprising alcohol and an organic phase rich in alkyl acrylate;
treatment of said organic phase rich in alkyl acrylate in a fourth distillation column to separate:
at the top of the fourth distillation column, purified alkyl acrylate and,
at the bottom of the fourth distillation column, heavy products and the polymerization inhibitors, and
continuous treatment of the bottom stream of the fourth distillation column, by evaporation, and at least two successive condensations of a gas phase resulting from the evaporation, to obtain:
an alkyl acrylate stream;
an alkyl alkoxypropionate stream, and
a residue containing the polymerization inhibitors.

2. The process as claimed in claim 1, in which the azeotropic distillation column comprises a sidestream drawoff of the organic phase rich in alkyl acrylate.

3. The process as claimed in claim 1, in which the azeotropic distillation is carried out by two distillation columns in series.

4. The process as claimed in claim 1, in which the evaporation of the bottom stream of the fourth distillation column is carried out in a temperature range extending from 80° C. to 110° C.

5. The process as claimed in claim 1, in which the maintained pressure of the evaporator is between 10,000 Pa and 80,000 Pa.

6. The process as claimed in claim 1, in which the azeotropic distillation is carried out under a slight vacuum at a pressure ranging from 200 to 600 mmHg.

7. The process as claimed in claim 1, in which said fourth distillation column for the purification of the alkyl acrylate comprises a tailing column and a topping column.

8. The process as claimed in claim 1, in which said fourth distillation column for the purification of the alkyl acrylate comprises a dividing wall column.

9. The process as claimed in claim 6, in which said fourth distillation column for the purification of the alkyl acrylate comprises a column having sidestream drawoff.

10. The process as claimed in claim 1, in which the top stream from the evaporator is cooled in two successive steps:
partial condensation in a temperature range extending from 70° C. to 110° C., at the same pressure as that of the evaporator, to obtain a liquid stream of Michael adducts to be purged and a vapor stream;
condensation of the vapor stream in a temperature range extending from 20° C. to 50° C. before the liquid stream is taken up by a pump and fed to the purification column.

11. The process as claimed in claim 1, the fourth distillation column comprising two distillation columns in series.

12. The process as claimed in claim 1, in which the alcohol is methanol, the alkyl acrylate is methyl acrylate, and the alkyl alkoxypropionate is methyl methoxypropionate.

13. The process as claimed in claim 12, in which methyl acrylate having a purity of greater than 99.5% by weight is obtained from the fourth distillation column operating in a pressure range from 10,000 Pa to 50,000 Pa and a temperature range extending from 80° C. to 110° C.

14. The process as claimed in claim 1, in which the alcohol is ethanol, the alkyl acrylate is ethyl acrylate, and the alkyl alkoxypropionate is ethyl ethoxypropionate.

15. The process as claimed in claim 1, in which the evaporation is carried out by means of a thin film evaporator.

16. The process as claimed in claim 1, further comprising a treatment by distillation of said aqueous phase generated by said liquid/liquid extraction, in a second distillation column, in order to separate:
at the top of the second distillation column, a top stream comprising alcohol, which is recycled in the esterification reaction, and
at the bottom of the second distillation column, an alcohol-depleted bottom stream.

17. The process as claimed in claim 1, further comprising a treatment of the bottom stream from said first distillation column, using a third distillation column and/or an evaporator which separates:
at the top of the distillation column and/or the evaporator, a stream comprising residual acrylic acid and lighter products, and
at the bottom of the distillation column and/or the evaporator, a stream consisting of heavy products.

18. The process as claimed in claim 1, wherein the alkyl acrylate stream generated by said continuous treatment of the bottom stream of the fourth distillation column, by evaporation, and at least two successive condensations of a gas phase resulting from the evaporation is recycled to the purification column.
